(19) United States
(12) Patent Application Publication
(10) Pub. No.: US 2022/0315876 A1
(43) Pub. Date: Oct. 6, 2022

(54) METHOD AND SYSTEM FOR STORING ENERGY IN THE FORM OF BIOPOLYMERS

(71) Applicant: LanzaTech, Inc., Skokie, IL (US)

(72) Inventors: Sean Dennis Simpson, Evanston, IL (US); Nicholas Bourdakos, Toronto (CA); Xueliang Li, Morton Grove, IL (US)

(21) Appl. No.: 17/656,462

(22) Filed: Mar. 25, 2022

Related U.S. Application Data
(60) Provisional application No. 63/171,032, filed on Apr. 5, 2021.

(51) Int. Cl. C12M 1/00; C08G 63/06; C12N 1/20; C12P 7/625; C25B 1/02; C25B 1/23; C25B 15/08

(52) U.S. Cl. CPC C12M 21/12; C08G 63/06; C12M 43/04; C12M 47/10; C12N 1/20; C12P 7/625; C25B 1/02; C25B 1/23; C25B 15/081

(57) ABSTRACT

The disclosure provides for methods and a system for storing energy in the form of a biopolymer. The method comprises intermittently processing electric energy generated from a renewable and/or non-renewable energy source in an electrolysis process to produce at least H₂, Oₒᵣ CO; intermittently passing H₂, O₂, or CO from the electrolysis process to a bioreactor containing a bacterial culture capable of producing a biopolymer; and fermenting the culture. The disclosure further provides a system for storing energy in the form of biopolymer comprising an electrolysis process in intermittent fluid communication with a renewable and/or non-renewable energy source for producing at least one of H₂, O₂, or CO; a bioreactor, in intermittent fluid communication with the electrolysis process and/or in continuous fluid communication with an industrial plant, comprising a reaction vessel suitable for intermittently growing, fermenting, and/or culturing and housing a microorganism capable of producing a biopolymer.

### CROSS REFERENCE TO RELATED APPLICATIONS

This application claims the benefit of U.S. Provisional Patent Application No. 63/171,032, filed Apr. 5, 2021, the entirety of which is incorporated herein by reference.

### FIELD

The disclosure relates to methods and systems for storing energy in the form of biopolymers and for improving the economics of a gas fermentation process. In particular, the disclosure relates to the combinations of a fermentation process with an industrial process, syngas process, and/or an electrolysis process where gases produced from the industrial process, syngas process, and/or electrolysis process are intermittently passed to a bioreactor for fermentation.

### BACKGROUND

Carbon dioxide (CO₂) accounts for about 76% of global greenhouse gas emissions from human activities, with methane (16%), nitrous oxide (6%), and fluorinated gases (2%) accounting for the balance (United States Environmental Protection Agency). Reduction of greenhouse gas emissions, particularly CO₂, is critical to halt the progression of global warming and the accompanying shifts in climate and weather.

It has long been recognized that catalytic processes, such as the Fischer-Tropsch process, may be used to convert gases containing carbon dioxide (CO₂), carbon monoxide (CO), and/or hydrogen (H₂), into a variety of fuels and chemicals. Recently, however, gas fermentation has emerged as an alternative platform for the biological fixation of such gases.

Such gases may be derived, for example, from industrial processes, including gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming).

With particular industrial or syngas processes the supply of gas may be insufficient for the fermentation process. When the supply of gas becomes insufficient for the fermentation process, the production rate of the fermentation process is less than optimal resulting in less products produced than what the fermentation process would otherwise be capable of producing.

Additionally, with a constantly adjusting market, the value of the products produced by the gas fermentation process varies. When the value of the products produced by the gas fermentation are high in comparison with the cost of producing such products, it is advantageous to increase the production rate of the fermentation process. In contrast, most renewable energy sources are intermittent, not transportable, and largely dependent on the meteorological and geographical conditions. This is particularly important for places which have a high energy demand, but are restricted to a seasonally fluctuating supply of renewable energies, such as solar or wind energy.

By increasing the production rate of the fermentation process at times when the market value of such products is high relative to the cost of producing such products, the economics of the fermentation process may be optimized with energy storage.

Many compounds have been assumed to act as storage materials in bacteria. Some of those compounds implicated as carbon and energy reserves are intracellular polysaccharides, particularly polyhydroxyalkanoates. Polyhyroxyalkanoates (PHA), particularly polyhyroxybutyrates (PHBs), accumulate in prokaryotes and serve as intracellular storage compounds for carbon and energy. Due to their thermoplastic characteristics and biodegradability, PHAs have various applications in industry and medicine.

There is still a need for a method and system which provides energy from a renewable or non-renewable energy source in a storable and transportable form that is also inexpensive, has high energy conversion rates and is environmentally friendly and sustainable.

Accordingly, there remains a need for improved integration of fermentation processes and energy storage with industrial, syngas processes, and/or electrolysis processes where the problems associated with the supply of feedstock are curtailed and the fermentation process can produce at maximum levels at times when such production is economically optimal.

### BRIEF SUMMARY

The disclosure provides a method for storing energy in the form of a biopolymer comprising intermittently processing at least a portion of electric energy generated from a renewable and/or non-renewable energy source in an electrolysis process to produce at least H₂, O₂ or CO; intermittently passing at least one of H₂, O₂, or CO from the electrolysis process to a bioreactor containing a culture comprising a liquid nutrient medium and a microorganism capable of producing a biopolymer; and fermenting the culture.

The disclosure also provides a system for storing energy in the form of biopolymer comprising an electrolysis process in intermittent fluid communication with a renewable and/or non-renewable energy source for producing at least one of H₂, O₂, or CO; an industrial plant for producing at least C1 feedstock; a bioreactor, in intermittent fluid communication with the electrolysis process and/or in continuous fluid communication with the industrial plant, comprising a reaction vessel suitable for intermittently growing, fermenting, and/or culturing and housing a microorganism capable of producing a biopolymer.

The disclosure provides a method for improving the performance and/or the economics of a fermentation process, the fermentation process defining a bioreactor containing a bacterial culture in a liquid nutrient medium, wherein the method comprises passing a C1 feedstock comprising one or both of CO and CO₂ from an industrial process to the bioreactor, wherein the C1 feedstock has a cost per unit, intermittently passing at least one of H₂, O₂, or CO from the electrolysis process to the bioreactor, wherein the electrolysis process has a cost per unit, and fermenting the culture to produce one or more fermentation products, wherein each of the one or more fermentation products has a value per unit. In certain instances, multiple electrolysis processes are utilized in order to provide one or all of CO, CO₂, and H₂ to the bioreactor.

In certain instances, the C1 feedstock is derived from an industrial or syngas process selected from the group comprising: gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). In certain instances, the C1 feedstock is derived from a combination of two or more sources. In certain instances, the C1 feedstock may further comprise H₂.

In one embodiment, the substrate comprises an industrial waste gas. In certain embodiments, the gas is steel mill waste gas or syngas.

In certain instances, the electrolysis process comprises CO. The electrolysis process comprising CO is derived from the electrolysis process of a CO₂-containing gaseous substrate. The CO₂-containing gaseous substrate may be derived from any gas stream containing CO₂. In particular instances, this CO₂-containing gas stream is derived at least in part from the group comprising: gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). In particular instances, the CO₂-containing gaseous substrate is derived from a combination of two or more sources.

In certain instances, the electrolysis process comprises H₂. The electrolysis process comprising H₂ is derived from the electrolysis process of water (H₂O). This water may be obtained from numerous sources. In various instances, the water may be obtained from the industrial process and/or the fermentation process. In various instances, the water may be obtained from a waste water treatment process. In particular instances, the water is obtained from a combination of two or more sources.

In particular instances, the disclosure improves the economics of the fermentation process by displacing at least a portion of the C1 feedstock from the industrial process with an electrolysis process. In various instances when the electrolysis process comprises H₂, the electrolysis process displaces at least a portion of the C1 feedstock from the industrial process as a means to adjust the molar ratio of H₂:CO:CO₂ of the feedstock being passed to the fermentation process. In certain instances, the electrolysis process comprising H₂ increases the molar ratio of H₂ in the feedstock being passed to the fermentation process.

The displacement of the C1 feedstock from the industrial process with an electrolysis process may be completed, at least in part, as a function of the cost per unit of the C1 feedstock and the cost per unit of the electrolysis process. In certain instances, the electrolysis process displaces at least a portion of the C1 feedstock when the cost per unit of electrolysis process is less than the cost per unit of C1 feedstock.

In particular instances, the disclosure improves the economics of the fermentation process by supplementing at least a portion of the C1 feedstock from the industrial process with electrolysis process. The supplementing of the C1 feedstock with the electrolysis process may be completed, at least in part, when the supply of the C1 feedstock is insufficient for the fermentation process.

In certain instances, the electrolysis process supplements at least a portion of the C1 feedstock as a function of the cost per unit of the electrolysis process and the value per unit of the fermentation product.

In certain instances, the electrolysis process supplements at least a portion of the C1 feedstock as a function of the cost per unit of the C1 feedstock the cost per unit of the electrolysis process, and the value per unit of the fermentation product.

In certain instances, the electrolysis process supplements the C1 feedstock when the cost per unit of the electrolysis process is less than the value per unit of the fermentation product. The cost per unit of electrolysis process may be less than the value per unit of the fermentation product when the cost of electricity is reduced. In certain instances, the cost of electricity is reduced due to the electricity being sourced from a renewable energy source. In certain instances, the renewable energy source is selected from the group consisting of solar, hydro, wind, geothermal, biomass, nitrogen, and nuclear.

The supplementing of the C1 feedstock comprising CO₂ with electrolysis process comprising H₂ may result in a number of benefits, including but not limited to, increasing the amount of CO₂ fixed in the one or more fermentation products. Therefore, in various instances, electrolysis process comprising H₂ supplements the C1 feedstock comprising CO₂ so as to increase the amount of CO₂ fixed in the one or more fermentation products.

In particular instances, the C1 feedstock contains proportions of various constituents that necessitate removal. In these instances, the C1 feedstock is treated to remove one or more constituent prior to passing the C1 feedstock to the bioreactor. The constituents removed from the C1 feedstock may be selected from the group comprising: sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, and tars.

In particular instances, the electrolysis process contains proportions of various constituents that necessitate removal. In these instances, the electrolysis process is treated to remove one or more constituent prior to passing the electrolysis process to the bioreactor. The constituents removed from the electrolysis process may be selected from the group comprising: sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, and tars. In particular instances at least one constituent removed from the electrolysis process comprises oxygen. At least one of the constituents removed may be produced, introduced, and/or concentrated by the electrolysis process. For example, oxygen may be produced, introduced, and/or concentrated by the electrolysis process of carbon dioxide. In various instances, oxygen is a by-product of the electrolysis process. In particular embodiments, oxygen is produced and/or concentrated in the electrolysis process.

Oxygen is a microbe inhibitor for many bacterial cultures. As such, oxygen may be inhibiting to the downstream fermentation process. In order to pass a non-inhibiting gas stream to the bioreactor where it may be fermented, at least a portion of oxygen, or other constituent, may need to be removed from the electrolysis process by one or more removal module.

In certain instances, the C1 feedstock is intermittently passed to the fermentation process at pressure. In these instances, the C1 feedstock from the industrial process is passed to one or more pressure module prior to being passed to the bioreactor for fermentation.

In certain instances, the electrolysis process is intermittently passed to the fermentation process at pressure. In these instances, the electrolysis process from the electrolysis process is passed to one or more pressure module prior to being passed to the bioreactor for fermentation.

Additionally, the electrolysis process may be completed at pressure. When completed at pressure, the material being electrolyzed is pressurized prior to being fed to the electrolysis process. In certain instances, the material being electrolyzed is a CO₂-containing gas stream. In instances where the CO₂-containing gas stream is pressurized prior to being electrolyzed, the CO₂-containing gas stream may be passed to a pressure module prior to being passed to the electrolysis process module.

In at least one embodiment, the method reduces the associated costs of producing various fermentation products. At least one of the one or more of the fermentation products may be ethanol, acetate, butyrate, 2,3-butanediol, lactate, butene, butadiene, ketones, methyl ethyl ketone, ethylene, acetone, isopropanol, lipids, 3-hydroypropionate, isoprene, fatty acids, 2-butanol, 1,2-propanediol, 1-propanol, and C6-C12 alcohols. At least one of the fermentation products may be further converted to at least one component of diesel, jet fuel, and/or gasoline.

In at least one embodiment, the method reduces the associated costs of producing various fermentation products. At least one of the one or more of the fermentation products may be selected from the group consisting of biopolymers, bioplastics, thermoplastics, microbial biomass, polyhydroxyalkanoates, or animal feed. At least one of the fermentation products may be further processed into at least one component of single cell protein and/or a cell-free protein synthesis platform by any method or combination of methods known in the art. In one embodiment, the polyhydroxyalkanoates may be converted into an end-product derived from polyhyroxyalkanoate.

In one embodiment, polyhydroxyalknaoates, poly-3-hyroxybutyrates, or poly-β-hydroxybutyrates occur in appreciable quantities in cells in the stationary phase when growth is limited by a deficiency of the supply of carbon and/or energy. In one embodiment, the carbon and/or energy source is intermittent.

In at least one embodiment, the methods and system of the disclosure provide that a cell will store any biopolymers or bioplastics that can be accumulated without decreasing the rate of growth. In one embodiment, the rate limiting factor in growth is the synthesis of proteins and nucleic acids when reserves containing H₂, O₂, and CO₂, accumulate, or in a primary degradative pathway of the carbon and energy source when no carbon and energy reserves accumulate. In another embodiment, the rate limiting factor in growth is the nature and level of nutrients in the medium.

At least one of the one or more fermentation products may be biomass produced by the culture. At least a portion of the microbial biomass may be converted to a single cell protein (SCP). At least a portion of the single cell protein may be utilized as a component of animal feed.

In one embodiment, the disclosure provides an animal feed comprising microbial biomass and at least one excipient, wherein the microbial biomass comprises a microorganism grown on a gaseous substrate comprising one or more of CO, CO₂, and H₂

In at least one embodiment, the electrolysis process is powered, at least in part, by a renewable energy source. In certain instances, the renewable energy source is selected from the group consisting of solar, hydro, wind, geothermal, biomass, nitrogen, and nuclear.

In certain embodiments, the industrial process may further produce a post-fermentation gaseous substrate. In various instances, this post-fermentation gaseous substrate comprises at least a portion of CO₂. In particular embodiments the post-fermentation gaseous substrate is passed to the electrolysis process.

In particular instances, the post-fermentation gaseous substrate contains proportions of various constituents that necessitate removal. In these instances, the post-fermentation gaseous substrate is treated to remove one or more constituent prior to passing the post-fermentation gaseous substrate to the electrolysis process. The constituents removed from the post-fermentation gaseous substrate may be selected from the group comprising: sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, and tars.

In particular instances at least one constituent removed from the post-fermentation gaseous substrate comprises sulphur. At least one of these constituents removed may be produced, introduced, and/or concentrated by the fermentation process. For example, sulphur, in the form of hydrogen sulfide (H₂S) may be produced, introduced, and/or concentrated by the fermentation process. In particular embodiments, hydrogen sulfide is introduced in the fermentation process. In various embodiments, the post-fermentation gaseous substrate comprises at least a portion of hydrogen sulfide. Hydrogen sulfide may be a catalyst inhibitor. As such, the hydrogen sulfide may be inhibiting to particular electrolysis processers. In order to pass a non-inhibiting post-fermentation gaseous substrate to the electrolysis processer at least a portion of the hydrogen sulfide, or other constituent present in the post-fermentation gaseous substrate, may need to be removed by one or more removal module.

In various embodiments, the constituent removed from the post-fermentation gaseous substrate, the industrial feedstock, and/or the electrolysis process is a microbe inhibitor and/or a catalyst inhibitor.

At least one removal module may be selected from the group comprising: hydrolysis module, acid gas removal module, deoxygenation module, catalytic hydrogenation module, particulate removal module, chloride removal module, tar removal module, and hydrogen cyanide removal module.

In certain instances, the electrolysis process may produce a carbon monoxide enriched stream and an oxygen enriched stream. In various instances, at least a portion of the separated carbon monoxide enriched stream may be passed to the bioreactor for fermentation. In some instances, the oxygen enriched stream may be passed to the industrial process to further improve the performance and/or economics of the industrial process.

In various embodiments where the electrolysis process comprises H₂, the H₂ may improve the fermentation substrate composition. Hydrogen provides energy required by the microorganism to convert carbon containing gases into useful products. When optimal concentrations of hydrogen are provided, the microbial culture can produce the desired fermentation products, for example ethanol, without the co-production of carbon dioxide.

The bacterial culture in the bioreactor comprises an autotrophic bacterium. In another embodiment the bacterial culture in the bioreactor comprises a hydrogenotrophic bacterium. The bacterium may be selected from the group consisting of Cupriavidus necator, Ralstonia eutropha, and Wautersia eutropha. In another embodiment, the bacterium may be selected from the group consisting of Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui.

In one particular embodiment, the parental microorganism is selected from the group of carboxydotrophic acetogenic bacteria, in one embodiment from the group comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui.

In one embodiment the parental microorganism is Clostridium autoethanogenum or Clostridium ljungdahlii. In one particular embodiment, the microorganism is Clostridium autoethanogenum DSM23693. In another particular embodiment, the microorganism is Clostridium ljungdahlii DSM13528 (or ATCC55383).

In one or more embodiment, the disclosure (i) decreases the cost associated with producing one or more fermentation product and/or (ii) increases the total amount of carbon converted to product, compared to a process without an electrolysis process.

In one embodiment, the disclosure provides a method and system for converting energy from any energy source, such as a local power grid, a renewable or non-renewable energy source, in an inexpensive way and with high process efficiency in a storable form as an end-product.

In another embodiment, the local power grid provides electricity intermittently passed as electrical energy produced by power based on availability of electrical power or the availability of electricity below a threshold price, where power prices fall as demand falls, or as set by the local power grid.

In one embodiment an autotrophic microorganism intermittently consumes, in part or entirely, the energy provided by the availability of power.

### BRIEF DESCRIPTION OF THE DRAWINGS

FIG. 1 is a plot of the gas uptake per litre of bioreactor liquid volume for the major gas components over the course of a 25-day continuous C. necator gas fermentation, with hydrogen as the energy source and CO₂ as the carbon source. The feed gas flow is lost at day 18.21 and recovered approximately 8 hours later. There is no significant change in the long-term stability of the fermentation, any fluctuations after gas recovery are within the normal operational fluctuations for this run.

FIG. 2 is a plot of the gas uptake per litre of bioreactor liquid volume for the major gas components over the course of the same 25-day continuous fermentation as in FIG. 1, with hydrogen as the energy source and CO₂ as the carbon source. This plot shows the gas outage in greater focus. The gas uptakes are almost immediately recovered after the resumption of the gas flow approximately 8 hours after the feed gas stopped flowing.

FIG. 3 is an example plot of stable biomass production for a C. necator gas fermentation, with hydrogen as the energy source and CO₂ as the carbon source. This plot shows continuous stable production over a 4.5-day period with an OD600 above 30 (equivalent to ˜30 g/L DCW C. necator biomass).

FIG. 4 is a plot of stable gas uptake per litre of bioreactor liquid volume for the major gas components in a C. necator gas fermentation, with hydrogen as the energy source and CO₂ as the carbon source. This plot shows continuous stable gas uptake over the same 4.5-day period as in FIG. 3.

FIG. 5 is a schematic flow diagram depicting the integration of an industrial process and an electrolysis process with a fermentation process.

### DETAILED DESCRIPTION

The following description of embodiments is given in general terms. The disclosure is further elucidated from the disclosure given under the heading “Examples” herein below, which provides experimental data supporting the disclosure, specific examples of various aspects of the disclosure, and means of performing the disclosure.

The inventors have identified that the integration of a gas fermentation process with an industrial process, syngas process, and/or an electrolysis process, where the electrolysis process intermittently supplies a fermentation process, and is capable of substantially improving the performance and/or economics of the fermentation process.

The inventors have surprisingly been able to turn on and off the feed source to the fermentation process with little to no start-up lag phase for the fermentation process. Further, the disclosure can be operated intermittently by storing energy in the form of a biopolymer, where product conversion can be intermittent during periods when an electricity grid is oversupplied with electricity, or idle when electricity is scarce or power is in demand. The disclosure provides a process that is capable of being fine-tuned to assist with balancing an electrical power grid system by storing energy in the form of a biopolymer.

Unless otherwise defined, the following terms as used throughout this specification are defined as follows:

The term “industrial process” refers to a process for producing, converting, refining, reforming, extracting, or oxidizing a substance involving chemical, physical, electrical, and/or mechanical steps. Exemplary industrial processes include, but are not limited to, carbohydrate fermentation, gas fermentation, cement making, pulp and paper making, steel making, oil refining and associated processes, petrochemical production, coke production, anaerobic or aerobic digestion, gasification (such as gasification of biomass, liquid waste streams, solid waste streams, municipal streams, fossil resources including natural gas, coal and oil), natural gas extraction, oil extraction, metallurgical processes, production and/or refinement of aluminum, copper, and/or ferroalloys, geological reservoirs, Fischer-Tropsch processes, methanol production, pyrolysis, steam methane reforming, dry methane reforming, partial oxidation of biogas or natural gas, and autothermal reforming of biogas or natural gas. In these embodiments, the substrate and/or C1-carbon source may be captured from the industrial process before it is emitted into the atmosphere, using any convenient method.

The term “electrolysis process”, may include any substrate leaving the electrolysis process. In various instances, the electrolysis process is comprised of CO, H₂, or combinations thereof. In certain instances, the electrolysis process may contain portions of unconverted CO₂. Preferably, the electrolysis process is fed from the electrolysis process to the fermentation process.

The terms “gas from an industrial process,” “gas source from an industrial process,” and “gaseous substrate from an industrial process” can be used interchangeably to refer to an off-gas from an industrial process, a by-product of an industrial process, a co-product of an industrial process, a gas recycled within an industrial process, and/or a gas used within an industrial facility for energy recovery. In some embodiments, a gas from an industrial process is a pressure swing adsorption (PSA) tail gas. In some embodiments, a gas from an industrial process is a gas obtained through a CO₂ extraction process, which may involve amine scrubbing or use of a carbonic anhydrase solution.

“C1” refers to a one-carbon molecule, for example, CO, CO₂, methane (CH₄), or methanol (CH₃OH). “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for a microorganism of the disclosure. For example, a C1-carbon source may comprise one or more of CO, CO₂, CH₄, CH₃OH, or formic acid (CH₂O₂). Preferably, a C1-carbon source comprises one or both of CO and CO₂. A “C1-fixing microorganism” is a microorganism that has the ability to produce one or more products from a C1-carbon source.

“Substrate” refers to a carbon and/or energy source. The substrate is gaseous and comprises a C1-carbon source, for example, CO, CO₂, and/or CH₄. Preferably, the substrate comprises a C1-carbon source of CO or CO and CO₂. The substrate may further comprise other non-carbon components, such as H₂, N₂, or electrons. As used herein, “substrate” may refer to a carbon and/or energy source for a microorganism of the disclosure. The substrate may refer to H₂ as the sole energy source.

The term “co-substrate” refers to a substance that, while not necessarily being the primary energy and material source for product synthesis, can be utilised for product synthesis when combined with another substrate, such as the primary substrate.

A “CO₂-comprising gaseous substrate,” “CO₂-comprising gas,” or “CO₂-comprising gaseous source” may include any gas that comprises CO₂. The gaseous substrate will comprise a significant proportion of CO₂, preferably at least about 5% to about 100% CO₂ by volume. Additionally, the gaseous substrate may comprise one or more of hydrogen (H₂), oxygen (O₂), nitrogen (N₂), and/or CH₄. As used herein, CO, H₂, and CH₄ may be referred to as “energy-rich gases.”

The term “carbon capture” as used herein refers to the sequestration of carbon compounds including CO₂ and/or CO from a stream comprising CO₂ and/or CO and either a) converting the CO₂ and/or CO into products, b) converting the CO₂ and/or CO into substances suitable for long term storage, c) trapping the CO₂ and/or CO in substances suitable for long term storage, or d) a combination of these processes.

The terms “increasing the efficiency,” “increased efficiency,” and the like refer to an increase in the rate and/or output of a reaction, such as an increased rate of converting the CO₂ and/or CO into products and/or an increased product concentration. When used in relation to a fermentation process, “increasing the efficiency” includes, but is not limited to, increasing one or more of the rate of growth of microorganisms catalysing a fermentation, the growth and/or product production rate at elevated product concentrations, the volume of desired product produced per volume of substrate consumed, the rate of production or level of production of the desired product, and the relative proportion of the desired product produced compared with other by-products of the fermentation.

“Reactant” as used herein refers to a substance that is present in a chemical reaction and is consumed during the reaction to produce a product. A reactant is a starting material that undergoes a change during a chemical reaction. In particular embodiments, a reactant includes, but is not limited to, CO and/or H₂. In particular embodiments, a reactant is CO₂. In one embodiment, a reactant is solely H₂.

A “CO-consuming process” refers to a process wherein CO is a reactant; CO is consumed to produce a product. A non-limiting example of a CO-consuming process is a C1-fixing gas fermentation process. A CO-consuming process may involve a CO₂-producing reaction. For example, a CO-consuming process may result in the production of at least one product, such as a fermentation product, as well as CO₂. In another example, acetic acid production is a CO-consuming process, wherein CO is reacted with methanol under pressure.

“Gas stream” refers to any stream of substrate which is capable of being passed, for example, from one module to another, from one module to a CO-consuming process, and/or from one module to a carbon capture means.

Gas streams typically will not be a pure CO₂ stream and will comprise proportions of at least one other component. For instance, each source may have differing proportions of CO₂, CO, H₂, and various constituents. Due to the varying proportions, a gas stream must be processed prior to being introduced to a CO-consuming process. Processing of the gas stream includes the removal and/or conversion of various constituents that may be microbe inhibitors and/or catalyst inhibitors. Preferably, catalyst inhibitors are removed and/or converted prior to being passed to an electrolysis module, and microbe inhibitors are removed and/or converted prior to being passed to a CO-consuming process. Additionally, a gas stream may need to undergo one or more concentration steps whereby the concentration of CO and/or CO₂ is increased. Preferably, a gas stream will undergo a concentration step to increase the concentration of CO₂ prior to being passed to the electrolysis module. It has been found that higher concentrations of CO₂ being passing into the electrolysis module results in higher concentrations of CO coming out of the electrolysis module.

The term “C1 feedstock”, may include any substrate leaving the industrial process. In various instances, the C1 feedstock is comprised of CO, H₂, CO₂, or combinations thereof. Preferably, the C1 feedstock is fed from the industrial process to the fermentation process.

The terms “improving the economics”, “optimizing the economics” and the like, when used in relationship to a fermentation process, include, but are not limited to, the increase of the amount of one or more of the products produced by the fermentation process during periods of time in which the value of the products produced is high relative to the cost of producing such products. The economics of the fermentation process may be improved by way of increasing the supply of feedstock to the bioreactor, which may be achieved for instance by supplementing the C1 feedstock from the industrial process with electrolysis process from the electrolysis process. The additional supply of feedstock may result in the increased efficiency of the fermentation process. Another means of improving the economics of the fermentation process is to select feedstock based upon the relative cost of the feedstock available. For example, when the cost of the C1 feedstock from the industrial process is higher than the cost of the electrolysis process from the electrolysis process, the electrolysis process may be utilized to displace at least a portion of the C1 feedstock By selecting feedstock based upon the cost of such feedstock the cost of producing the resulting fermentation product is reduced.

The electrolysis process is capable of supplying feedstock comprising one or both of H₂ and CO. The “cost per unit of electrolysis process” may be expressed in terms of any given product produced by the fermentation process and any electrolysis process, for example for the production of ethanol with the electrolysis process defined as H₂, the cost per unit of electrolysis process is defined by the following equation:

$$( \frac{\$ z}{MWh} ) \times ( \frac{1 MWh}{3.6 G {J}_{electricity}} ) \times ( x \frac{{GJ}_{electricity}}{{GJ}_{H 2}} ) \times ( y \frac{{GJ}_{H 2}}{G {J}_{ethanol}} )$$

where z represents the cost of power, x represents the electrolysis process efficiency, and y represents the yield of ethanol.

For the production of ethanol with electrolysis process defined as CO, the cost per unit of electrolysis process is defined by the following equation:

$$( \frac{\$ z}{MWh} ) \times ( \frac{1 MWh}{3.6 {GJ}_{electricity}} ) \times ( x \frac{{GJ}_{electricity}}{{GJ}_{CO}} ) \times ( y \frac{{GJ}_{CO}}{{GJ}_{ethanol}} )$$

where z represents the cost of power, x represents the electrolysis process efficiency, and y represents the yield of ethanol.

In addition to the cost of feedstock, the fermentation process includes “production costs.” The “production costs” exclude the cost of the feedstock. “Production costs”, “marginal cost of production”, and the like, include the variable operating costs associated with running the fermentation process. This value may be dependent on the product being produced. The marginal cost of production may be represented by a fixed cost per unit of product, which may be represented in terms of the heating value of combustion of the product. For example, the calculation of the marginal cost of production for ethanol is defined by the following equation:

$$( \frac{\$ c}{metric ton} ) \times ( \frac{1 metric ton}{26.8 {GJ}_{ethanol}} )$$

where c represents the variable operating costs associated with running the bioreactor and 26.8 GJ represents the lower heating value of combustion of ethanol. In certain instances, the variable operating costs associated with running the bioreactor, c, is $200 for ethanol excluding the price of H₂/CO/CO₂.

The fermentation process is capable of producing a number of products. Each product defining a different value. The “value of the product” may be determined based upon the current market price of the product and the heating value of combustion of the product. For example, the calculation for the value of ethanol is defined by the following equation:

$$( \frac{\$ z}{metric ton} ) \times ( \frac{1 metric ton}{26.8 {GJ}_{ethanol}} )$$

where z is the current value of ethanol per metric ton and 26.8 GJ represents the lower heating value of combustion of ethanol.

To optimize the economics of the fermentation process, the value of the product produced must exceed the “cost of producing” such product. The cost of producing a product is defined as the sum of the “cost of feedstock” and the “marginal cost of production.” The economics of the fermentation process may be expressed in terms of a ratio defined by the value of product produced compared to the cost of producing such product. The economics of the fermentation process is improved as the ratio of the value of the product compared to the cost of producing such product increases. The economics of the fermentation process may be dependent on the value of the product produced, which may change dependent, at least in part, on the fermentation process implemented, including but not limited to the bacterial culture and/or the composition of the gas used in the fermentation process. When ethanol is the product produced by the fermentation process the economics may be determined by the following ratio:

$$( \frac{\$ z}{G {J}_{e t h a n o l}} ) : ( \frac{\$ x}{G {J}_{e t h a n o l}} ) + ( \frac{\$ y}{G {J}_{e t h a n o l}} )$$

where z represents the value of ethanol, x represents the cost of feedstock, and y represents the marginal cost of production (excluding feedstock).

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated product concentrations, the volume of desired product produced per volume of substrate consumed, the rate of production or level of production of the desired product, and the relative proportion of the desired product produced compared with other by-products of the fermentation. In certain instances, the electrolysis process increases the efficiency of the fermentation process.

The term “insufficient” and the like, when used in relation to the supply of feedstock for the fermentation process, includes, but is not limited to, lower than optimal amounts, whereby the fermentation process produces less quantity of fermentation product than the fermentation process otherwise would had the fermentation process been supplied with higher amounts of feedstock. For example, the supply of feedstock may become insufficient at times when the industrial process is not providing enough C1 feedstock to adequately supply the fermentation process. Preferably, the fermentation process is supplied with optimal amounts of feedstock such that the quantity of fermentation product is not limited by the feedstock supply.

“C1-containing gaseous substrate” may include any gas which contains one or both of carbon dioxide and carbon monoxide. The gaseous substrate will contain a significant proportion of CO₂, preferably at least about 5% to about 100% CO₂ by volume. Additionally, the gaseous substrate may contain one or more of hydrogen (H₂), oxygen (O₂), nitrogen (N₂), and/or methane (CH₄).

“Concentration module” and the like refer to technology capable of increasing the level of a particular component in a gas stream. In particular embodiments, the concentration module is a CO₂ concentration module, wherein the proportion of CO₂ in the gas stream leaving the CO₂ concentration module is higher relative to the proportion of CO₂ in the gas stream prior to being passed to the CO₂ concentration module. In some embodiments, a CO₂ concentration module uses deoxygenation technology to remove O₂ from a gas stream and thus increase the proportion of CO₂ in the gas stream. In some embodiments, a CO₂ concentration module uses pressure swing adsorption (PSA) technology to remove H₂ from a gas stream and thus increase the proportion of CO₂ in the gas stream. In certain instances, a fermentation process performs the function of a CO₂ concentration module. In some embodiments, a gas stream from a concentration module is passed to a carbon capture and sequestration (CCS) unit or an enhanced oil recovery (EOR) unit.

The terms “electrolysis module” and “electrolyzer” can be used interchangeably to refer to a unit that uses electricity to drive a non-spontaneous reaction. Electrolysis technologies are known in the art. Exemplary processes include alkaline water electrolysis, proton or anion exchange membrane (PEM, AEM) electrolysis, and solid oxide electrolysis (SOE) (Ursua et al., Proceedings of the IEEE 100(2):410-426, 2012; Jhong et al., Current Opinion in Chemical Engineering 2:191-199, 2013). The term “faradaic efficiency” is a value that references the number of electrons flowing through an electrolyzer and being transferred to a reduced product rather than to an unrelated process. SOE modules operate at elevated temperatures. Below the thermoneutral voltage of an electrolysis module, an electrolysis reaction is endothermic. Above the thermoneutral voltage of an electrolysis module, an electrolysis reaction is exothermic. In some embodiments, an electrolysis module is operated without added pressure. In some embodiments, an electrolysis module is operated at a pressure of 5-10 bar.

A “CO₂ electrolysis module” refers to a unit capable of splitting CO₂ into CO and O₂ and is defined by the following stoichiometric reaction: 2CO₂+electricity→2CO+O₂. The use of different catalysts for CO₂ reduction impact the end product. Catalysts including, but not limited to, Au, Ag, Zn, Pd, and Ga catalysts, have been shown effective for the production of CO from CO₂. In some embodiments, the pressure of a gas stream leaving a CO₂ electrolysis module is approximately 5-7 bar.

“H₂ electrolysis module,” “water electrolysis module,” and “H₂O electrolysis module” refer to a unit capable of splitting H₂O, in the form of steam, into H₂ and O₂ and is defined by the following stoichiometric reaction: 2H₂O+electricity→2H₂+O₂. An H₂O electrolysis module reduces protons to H₂ and oxidizes O²⁻ to O₂. H₂ produced by electrolysis can be blended with a C1-comprising gaseous substrate as a means to supply additional feedstock and to improve substrate composition.

H₂ and CO₂ electrolysis modules have 2 gas outlets. One side of the electrolysis module, the anode, comprises H₂ or CO (and other gases such as unreacted water vapor or unreacted CO₂). The second side, the cathode, comprises O₂ (and potentially other gases). The composition of a feedstock being passed to an electrolysis process may determine the presence of various components in a CO stream. For instance, the presence of inert components, such as CH₄ and/or N₂, in a feedstock may result in one or more of those components being present in the CO-enriched stream. Additionally, in some electrolyzers, O₂ produced at the cathode crosses over to the anode side where CO is generated and/or CO crosses over to the anode side, leading to cross contamination of the desired gas products.

The term “separation module” is used to refer to a technology capable of dividing a substance into two or more components. For example, an “O₂ separation module” may be used to separate an O₂-comprising gaseous substrate into a stream comprising primarily O₂ (also referred to as an “O₂-enriched stream” or “O₂-rich gas”) and a stream that does not primarily comprise O₂, comprises no O₂, or comprises only trace amounts of O₂ (also referred to as an “O₂-lean stream” or “O₂-depleted stream”).

As used herein, the terms “enriched stream,” “rich gas,” “high purity gas,” and the like refer to a gas stream having a greater proportion of a particular component following passage through a module, such as an electrolysis module, as compared to the proportion of the component in the input stream into the module. For example, a “CO-enriched stream” may be produced upon passage of a CO₂-comprising gaseous substrate through a CO₂ electrolysis module. An “H₂-enriched stream” may be produced upon passage of a water gaseous substrate through an H₂ electrolysis module. An “O₂-enriched stream” emerges automatically from the anode of a CO₂ or H₂ electrolysis module; an “O₂-enriched stream” may also be produced upon passage of an O₂-comprising gaseous substrate through an O₂ separation module. A “CO₂-enriched stream” may be produced upon passage of a CO₂-comprising gaseous substrate through a CO₂ concentration module.

As used herein, the terms “lean stream,” “depleted gas,” and the like refer to a gas stream having a lesser proportion of a particular component following passage through a module, such as a concentration module or a separation module, as compared to the proportion of the component in the input stream into the module. For example, an O₂-lean stream may be produced upon passage of an O₂-comprising gaseous substrate through an O₂ separation module. The O₂-lean stream may comprise unreacted CO₂ from a CO₂ electrolysis module. The O₂-lean stream may comprise trace amounts of O₂ or no O₂. A “CO₂-lean stream” may be produced upon passage of a CO₂-comprising gaseous substrate through a CO₂ concentration module. The CO₂-lean stream may comprise CO, H₂, and/or a constituent such as a microbe inhibitor or a catalyst inhibitor. The CO₂-lean stream may comprise trace amounts of CO₂ or no CO₂.

In embodiments, the disclosure provides an integrated process wherein the pressure of the gas stream is capable of being increased and/or decreased. The term “pressure module” refers to a technology capable of producing (i.e., increasing) or decreasing the pressure of a gas stream. The pressure of the gas may be increased and/or decreased through any suitable means, for example one or more compressor and/or valve. In certain instances, a gas stream may have a lower than optimum pressure, or the pressure of the gas stream may be higher than optimal, and thus, a valve may be included to reduce the pressure. A pressure module may be located before or after any module described herein. For example, a pressure module may be utilized prior to a removal module, prior to a concentration module, prior to an electrolysis module, and/or prior to a CO-consuming process.

A “pressurized gas stream” refers to a gaseous substrate that has passed through a pressure module. A “pressurized gas stream” may also be used to refer to a gas stream that meets the operating pressure requirements of a particular module.

The terms “post-CO-consuming process gaseous substrate,” “post-CO-consuming process tail gas,” “tail gas,” and the like may be used interchangeably to refer to a gas that has passed through a CO-consuming process. The post-CO-consuming process gaseous substrate may comprise unreacted CO, unreacted H₂, and/or CO₂ produced (or not taken up in parallel) by the CO-consuming process. The post-CO-consuming process gaseous substrate may further be passed to one or more of a pressure module, a removal module, a CO₂ concentration module, and/or an electrolysis module. In some embodiments, a “post-CO-consuming process gaseous substrate” is a post-fermentation gaseous substrate.

The term “desired composition” is used to refer to the desired level and types of components in a substance, such as, for example, of a gas stream. More particularly, a gas is considered to have a “desired composition” if it contains a particular component (i.e., CO, H₂, and/or CO₂) and/or contains a particular component at a particular proportion and/or does not comprise a particular component (i.e., a contaminant harmful to the microorganisms) and/or does not comprise a particular component at a particular proportion. More than one component may be considered when determining whether a gas stream has a desired composition.

While it is not necessary for the substrate to comprise any H₂, the presence of H₂ should not be detrimental to product formation in accordance with methods of the disclosure. In particular embodiments, the presence of H₂ results in an improved overall efficiency of alcohol production. In one embodiment, the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially H₂ free.

The substrate may also contain some CO for example, such as about 1% to about 80% CO by volume, or 1% to about 30% CO by volume. In one embodiment, the substrate comprises less than or equal to about 20% CO by volume. In particular embodiments, the substrate comprises less than or equal to about 15% CO by volume, less than or equal to about 10% CO by volume, less than or equal to about 5% CO by volume or substantially no CO.

Substrate composition can be improved to provide a desired or optimum H₂:CO:CO₂ molar ratio. The desired H₂:CO:CO₂ molar ratio is dependent on the desired fermentation product of the fermentation process. For ethanol, the optimum H₂:CO:CO₂ molar ratio would be: (x):(y):

$$( \frac{x - 2 y}{3} ) ,$$

where x>2y, in order to satisfy the molar stoichiometry for ethanol production

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} O .$$

Operating the fermentation process in the presence of hydrogen, has the added benefit of reducing the amount of CO₂ produced by the fermentation process. For example, a gaseous substrate comprising minimal H₂, will produce ethanol and CO₂ by the following molar stoichiometry [6 CO+3H₂O→C₂H₅OH+4 CO₂]. As the amount of hydrogen utilized by the C1 fixing bacterium increases, the amount of CO₂ produced decreases [i.e., 2 CO+4H₂→C₂H₅OH+H₂O].

When CO is the sole carbon and energy source for ethanol production, a portion of the carbon is lost to CO₂ as follows:

6 CO+3H₂O→C₂H₅OH+4 CO₂(ΔG°=−224.90 kJ/mol ethanol)

As the amount of H₂ available in the substrate increases, the amount of CO₂ produced decreases. At a molar stoichiometric ratio of 1:2 (CO/H₂), CO₂ production is completely avoided.

5 CO+1H₂+2 H₂O→1 C₂H₅OH+3 CO₂(ΔG°=−204.80 kJ/mol ethanol)

4 CO+2H₂+1 H₂O→1 C₂H₅OH+2 CO₂(ΔG°=−184.70 kJ/mol ethanol)

3 CO+3H₂→1 C₂H₅OH+1 CO₂(ΔG°=−164.60 kJ/mol ethanol)

“Gas stream” refers to any stream of substrate which is capable of being passed, for example, from one module to another, from one module to a bioreactor, from one process to another process, and/or from one module to a carbon capture means.

“Reactants” as used herein refer to a substance that takes part in and undergoes change during a chemical reaction. In particular embodiments, the reactants include, but are not limited to, CO and/or H₂.

“Microbe inhibitors” as used herein refer to one or more constituent that slows down or prevents a particular chemical reaction or other process including the microbe. In particular embodiments, the microbe inhibitors include, but are not limited to, Oxygen (O₂), hydrogen cyanide (HCN), acetylene (C₂H₂), and BTEX (benzene, toluene, ethyl benzene, xylene).

“Catalyst inhibitor”, “adsorbent inhibitor”, and the like, as used herein, refer to one or more substance that decreases the rate of, or prevents, a chemical reaction. In particular embodiments, the catalyst and/or adsorbent inhibitors may include, but are not limited to, hydrogen sulfide (H₂S) and carbonyl sulfide (COS).

“Removal module”, “clean-up module”, “processing module” and the like includes technologies that are capable of either converting and/or removing microbe inhibitors, and/or catalyst inhibitors from the gas stream.

The term “constituents”, “contaminants”, and the like, as used herein, refers to the microbe inhibitors, and/or catalyst inhibitors that may be found in the gas stream. In particular embodiments, the constituents include, but are not limited to, sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, and tars. Preferably, the constituents removed by the removal module does not include carbon dioxide (CO₂).

The term “treated gas” refers to the gas stream that has been passed through at least one removal module and has had one or more constituent removed and/or converted.

The term “carbon capture” as used herein refers to the sequestration of carbon compounds including CO₂ and/or CO from a stream comprising CO₂ and/or CO and either:
converting the CO₂ and/or CO into products; or
converting the CO₂ and/or CO into substances suitable for long term storage; or
trapping the CO₂ and/or CO in substances suitable for long term storage;
or a combination of these processes.

The term “bioreactor” includes a fermentation device consisting of one or more vessels and/or towers or piping arrangements, which includes the Continuous Stirred Tank Reactor (CSTR), Immobilized Cell Reactor (ICR), Trickle Bed Reactor (TBR), Bubble Column, Gas Lift Fermenter, Static Mixer, a circulated loop reactor, a membrane reactor, such as a Hollow Fibre Membrane Bioreactor (HFM BR) or other vessel or other device suitable for gas-liquid contact. The reactor is preferably adapted to receive a gaseous substrate comprising CO or CO₂ or H₂ or mixtures thereof. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

“Nutrient media” or “Nutrient medium” is used to describe bacterial growth media. Generally, this term refers to a media containing nutrients and other components appropriate for the growth of a microbial culture. The term “nutrient” includes any substance that may be utilised in a metabolic pathway of a microorganism. Exemplary nutrients include potassium, B vitamins, trace metals and amino acids.

The term “fermentation broth” or “broth” is intended to encompass the mixture of components including nutrient media and a culture or one or more microorganisms. It should be noted that the term microorganism and the term bacteria are used interchangeably throughout the document.

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “desired composition” is used to refer to the desired level and types of components in a substance, such as, for example, of a gas stream. More particularly, a gas is considered to have a “desired composition” if it contains a particular component (i.e. CO, H₂, and/or CO₂) and/or contains a particular component at a particular proportion and/or does not contain a particular component (i.e. a constituent harmful to the microorganisms) and/or does not contain a particular component at a particular proportion. More than one component may be considered when determining whether a gas stream has a desired composition.

Unless the context requires otherwise, the phrases “fermenting”, “fermentation process” or “fermentation reaction” and the like, as used herein, are intended to encompass both the growth phase and product biosynthesis phase of the gaseous substrate.

A “microorganism” is a microscopic organism, especially a bacterium, archaea, virus, or fungus. The microorganism of the disclosure is typically a bacterium. As used herein, recitation of “microorganism” should be taken to encompass “bacterium.”

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism. In one embodiment, the parental microorganism is Cupriavidus necator, Clostridium autoethanogenum, Clostridium ljungdahlii, or Clostridium ragsdalei. In an embodiment, the parental microorganism is Clostridium autoethanogenum LZ1561, which was deposited on Jun. 7, 2010 with Deutsche Sammlung von Mikroorganismen and Zellkulturen GmbH (DSMZ) located at InhoffenstraBe 7B, D-38124 Braunschweig, Germany on Jun. 7, 2010 under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (i.e., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the disclosure is derived from a parental microorganism. In one embodiment, the microorganism of the disclosure is derived from Cupriavidus necator, Clostridium autoethanogenum, Clostridium ljungdahlii, or Clostridium ragsdalei. In an embodiment, the microorganism of the disclosure is derived from Clostridium autoethanogenum LZ1561, which is deposited under DSMZ accession number DSM23693.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

Metabolic engineering of microorganisms, such as Clostridia, can tremendously expand their ability to produce many important fuel and chemical molecules other than native metabolites, such as ethanol. However, until recently, Clostridia were considered genetically intractable and therefore generally off limits to extensive metabolic engineering efforts. In recent years several different methods for genome engineering for Clostridia have been developed including intron-based methods (ClosTron) (Kuehne, Strain Eng: Methods and Protocols, 389-407, 2011), allelic exchange methods (ACE) (Heap, Nucl Acids Res, 40: e59, 2012; Ng, PLoS One, 8: e56051, 2013), Triple Cross (Liew, Frontiers Microbiol, 7: 694, 2016), methods mediated through I-SceI (Zhang, Journal Microbiol Methods, 108: 49-60, 2015), MazF (Al-Hinai, Appl Environ Microbiol, 78: 8112-8121, 2012), or others (Argyros, Appl Environ Microbiol, 77: 8288-8294, 2011), Cre-Lox (Ueki, mBio, 5: e01636-01614, 2014), and CRISPR/Cas9 (Nagaraju, Biotechnol Biofuels, 9: 219, 2016). However, it remains extremely challenging to iteratively introduce more than a few genetic changes, due to slow and laborious cycling times and limitations on the transferability of these genetic techniques across species. Furthermore, we do not yet sufficiently understand C1 metabolism in Clostridia to reliably predict modifications that will maximize C1 uptake, conversion, and carbon/energy/redox flows towards product synthesis. Accordingly, introduction of target pathways in Clostridia remains a tedious and time-consuming process.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure. Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide”, “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

In particular, a “disruptive mutation” is a mutation that reduces or eliminates (i.e., “disrupts”) the expression or activity of a gene or enzyme. The disruptive mutation may partially inactivate, fully inactivate, or delete the gene or enzyme. The disruptive mutation may be any mutation that reduces, prevents, or blocks the biosynthesis of a product produced by an enzyme. The disruptive mutation may be a knockout (KO) mutation. The disruption may also be a knockdown (KD) mutation that reduces, but does not entirely eliminate, the expression or activity of a gene, protein, or enzyme. While KOs are generally effective in increasing product yields, they sometimes come with the penalty of growth defects or genetic instabilities that outweigh the benefits, particularly for non-growth coupled products. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

“Codon optimization” refers to the mutation of a nucleic acid, such as a gene, for optimized or improved translation of the nucleic acid in a particular strain or species. Codon optimization may result in faster translation rates or translation accuracy. In an embodiment, the genes of the disclosure are codon optimized for expression in Clostridium, particularly Clostridium autoethanogenum, Clostridium ljungdahlii, or Clostridium ragsdalei. In a further embodiment, the genes of the disclosure are codon optimized for expression in Clostridium autoethanogenum LZ1561, which is deposited under DSMZ accession number DSM23693.

“Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as Genbank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are well known in the art (e.g., Tijssen, Laboratory techniques in biochemistry and molecular biology-hybridization with nucleic acid probes, Second Chapter “Overview of principles of hybridization and the strategy of nucleic acid probe assay,” Elsevier, N.Y, 1993).

“Hybridization” refers to a reaction in which one or more polynucleotides react to form a complex that is stabilized via hydrogen bonding between the bases of the nucleotide residues. The hydrogen bonding may occur by Watson Crick base pairing, Hoogsteen binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In an embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate:ferredoxin oxidoreductase promoter, an Rnf complex operon promoter, an ATP synthase operon promoter, or a phosphotransacetylase/acetate kinase operon promoter.

“Wood-Ljungdahl” refers to the Wood-Ljungdahl pathway of carbon fixation as described, i.e., by Ragsdale, Biochim Biophys Acta, 1784: 1873-1898, 2008. “Wood-Ljungdahl microorganisms” refers, predictably, to microorganisms containing the Wood-Ljungdahl pathway. Generally, the microorganism of the disclosure contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (i.e., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1-carbon source.

An “anaerobe” is a microorganism that does not require oxygen for growth. An anaerobe may react negatively or even die if oxygen is present above a certain threshold. However, some anaerobes are capable of tolerating low levels of oxygen (i.e., 0.000001-5 vol % oxygen).

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, N.Y., 2006). All naturally occurring acetogens are C1-fixing, anaerobic, autotrophic, and non-methanotrophic. In one embodiment, the microorganism of the disclosure is an acetogen.

An “ethanologen” is a microorganism that produces or is capable of producing ethanol. In one embodiment, the microorganism of the disclosure is an ethanologen.

An “autotroph” is a microorganism capable of growing in the absence of organic carbon. Instead, autotrophs use inorganic carbon sources, such as CO and/or CO₂. Typically, the microorganism of the disclosure is an autotroph.

A “carboxydotroph” is a microorganism capable of utilizing CO as a sole source of carbon and energy.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

A “hydrogenotroph” is a microorganisms capable of utilizing H₂ as a sole source of energy. In certain embodiments, the microorganism of the disclosure is a hydrogenotroph or is derived from a hydrogenotroph.

“Substrate” refers to a carbon and/or energy source for the microorganism of the disclosure. The substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons.

The term “co-substrate” refers to a substance that, while not necessarily being the primary energy and material source for product synthesis, can be utilised for product synthesis when added to another substrate, such as the primary substrate.

The substrate and/or C1-carbon source may be a waste gas obtained as a by-product of an industrial process or from some other source, such as from automobile exhaust fumes or biomass gasification. In certain embodiments, the industrial process is selected from the group consisting gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). In various instances, the substrate and/or C1-carbon source may be captured from the industrial process before it is emitted into the atmosphere, using any convenient method.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In certain embodiments, the fermentation is performed in the absence of carbohydrate substrates, such as sugar, starch, lignin, cellulose, or hemicellulose.

The microorganism of the disclosure may be cultured with the gaseous substrate to produce one or more products. For instance, the microorganism of the disclosure may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and/or monoethylene glycol (WO 2019/126400) in addition to 2-phenylethanol. In certain embodiments, microbial biomass itself may be considered a product. These products may be further converted to produce at least one component of diesel, jet fuel, and/or gasoline. In certain embodiments, 2-phenylethanol may be used as an ingredient in fragrances, essential oils, flavors, and soaps. Additionally, the microbial biomass may be further processed to produce a single cell protein (c) by any method or combination of methods known in the art. In addition to one or more target products, the microorganism of the disclosure may also produce ethanol, acetate, and/or 2,3-butanediol.

A “single cell protein” (SCP) refers to a microbial biomass that may be used in protein-rich human and/or animal feeds, often replacing conventional sources of protein supplementation such as soymeal or fishmeal. To produce a single cell protein, or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

“Microbial biomass” refers biological material comprising microorganism cells. For example, microbial biomass may comprise or consist of a pure or substantially pure culture of a bacterium, archaea, virus, or fungus. When initially separated from a fermentation broth, microbial biomass generally contains a large amount of water. This water may be removed or reduced by drying or processing the microbial biomass.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavour, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3^{rd} revision, pages 575-633, 2014.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism, but is not produced by a genetically unmodified microorganism from which the genetically modified microorganism is derived.

A “biopolymer” refers to natural polymers produced by the cells of living organisms. In certain embodiments, the biopolymer is PHA. In certain embodiments, the biopolymer is PHB.

A “bioplastic” refers to plastic materials produced from renewable biomass sources. A bioplastic may be produced from renewable sources, such as vegetable fats and oils, corn starch, straw, woodchips, sawdust, or recycled food waste.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product account for at least about 5 wt. %, 10 wt. %, 15 wt. %, 20 wt. %, 30 wt. %, 50 wt. %, 75 wt. %, or 90 wt. % of all fermentation products produced by the microorganism of the disclosure. In one embodiment, the target product accounts for at least 10 wt. % of all fermentation products produced by the microorganism of the disclosure, such that the microorganism of the disclosure has a selectivity for the target product of at least 10 wt. %. In another embodiment, the target product accounts for at least 30 wt. % of all fermentation products produced by the microorganism of the disclosure, such that the microorganism of the disclosure has a selectivity for the target product of at least 30 wt. %. In one embodiment, the target product accounts for at least 90 wt. % of all fermentation products produced by the microorganisms, such that the microorganism of the disclosure has a selectivity for the target product of at least 90 wt. %.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably returned to the bioreactor. The cell-free permeate remaining after target products have been removed is also preferably returned to the bioreactor. Additional nutrients (such as B vitamins) may be added to the cell-free permeate to replenish the medium before it is returned to the bioreactor.

Carbon monoxide and oxygen can be produced by an electrolysis process, defined by the following molar stoichiometric reaction: 2CO₂+electricity→2CO+O₂. The carbon monoxide produced by electrolysis process can be used as a feedstock for gas fermentation. Additionally, it is considered that the produced CO can be used alongside feedstock from an industrial process, as a means to provide additional feedstock and/or improve the fermentation substrate composition.

The electrolysis process is also capable of producing hydrogen from water, defined by the following molar stoichiometric reaction: 2H₂O+electricity→2H₂+O₂. The hydrogen produced by electrolysis process can be used as a feedstock for gas fermentation. This hydrogen may be used alongside feedstock from an industrial process, as a means to provide additional feedstock and/or improve the fermentation substrate composition.

The use of the electrolysis process may be used at times when economically viable. In certain instances, the feedstock from the electrolysis process may increase the efficiency of the fermentation process by reducing the costs associated with production.

The CO₂-containing substrate utilized by the electrolysis process for producing carbon monoxide may be derived from a number of sources. The CO₂-containing gaseous substrate may be derived, at least in part, from any gas containing CO₂, selected from the group comprising: gas from carbohydrate fermentation, gas from cement making, pulp and paper making, steel making, oil refining and associated processes, petrochemical production, coke production, anaerobic or aerobic digestion, synthesis gas (derived from sources including but not limited to biomass, liquid waste streams, solid waste streams, municipal streams, fossil resources including natural gas, coal and oil), natural gas extraction, oil extraction, metallurgical processes, for production and/or refinement of aluminium, copper, and/or ferroalloys, geological reservoirs, and catalytic processes (derived from steam sources including but not limited to steam methane reforming, steam naphtha reforming, petroleum coke gasification, catalyst regeneration—fluid catalyst cracking, catalyst regeneration-naphtha reforming, and dry methane reforming). Additionally, the substrate may be captured from the industrial process before it is emitted into the atmosphere, using any conventional method. Furthermore, the CO₂-containing substrate may be derived from a combination of two or more of the above-mentioned sources.

Gas streams typically will not be a pure CO₂ stream, and will contain proportions of at least one other component. For instance, each source may have differing proportions of CO₂, CO, H₂, and various constituents. Due to the varying proportions, the gas stream may be processed prior to being introduced to the bioreactor and/or the electrolysis process module. The processing of the gas stream includes the removal and/or conversion of various constituents that may be microbe inhibitors and/or catalyst inhibitors. Preferably, the catalyst inhibitors are removed and/or converted prior to being passed to the electrolysis process module, and the microbe inhibitors are removed and/or converted prior to being passed to the bioreactor.

Typical constituents found in the gas stream that may need to be removed and/or converted include, but are not limited to, sulphur compounds, aromatic compounds, alkynes, alkenes, alkanes, olefins, nitrogen compounds, phosphorous-containing compounds, particulate matter, solids, oxygen, oxygenates, halogenated compounds, silicon containing compounds, carbonyls, metals, alcohols, esters, ketones, peroxides, aldehydes, ethers, and tars.

These constituents may be removed by conventional removal modules known in the art. These removal modules may be selected from the following: hydrolysis module, acid gas removal module, deoxygenation module, catalytic hydrogenation module, particulate removal module, chloride removal module, tar removal module, and hydrogen cyanide removal module.

In various embodiments, at least a portion of the electrolysis process may be sent to storage. Certain industrial processes may include storage means for long-term or short-term storage of gaseous substrates and/or liquid substrates. In instances where at least a portion of the electrolysis process is sent to storage, the electrolysis process may be sent to the same storage means utilized by the industrial process, for example an existing gas holder at a steel mill. At least a portion of the electrolysis process may be sent to independent storage means, where electrolysis process is stored separately from the C1 feedstock from the industrial process. In certain instances, this stored feedstock from one or both of the industrial process and/or the one or more electrolysis processes may be used by the fermentation process at a later time.

In various embodiments, the disclosure provides an integrated process comprising electrolysis process wherein the power supplied for the electrolysis process is derived, at least in part, from a renewable energy source. In certain instances, the renewable energy source is selected from the group consisting of solar, hydro, wind, geothermal, biomass, nitrogen, and nuclear.

Although the substrate is typically gaseous, the substrate may also be provided in alternative forms. For example, the substrate may be dissolved in a liquid saturated with a CO-containing gas using a microbubble dispersion generator. By way of further example, the substrate may be adsorbed onto a solid support.

In addition to increasing the efficiency of the fermentation process, the electrolysis process may increase the efficiency of the industrial process. The increase in efficiency of the industrial process may be achieved through use of an electrolysis process by-product, namely, oxygen. Specifically, the O₂ by-product of the electrolysis process may be used by the C1-generating industrial process. Many C1-generating industrial processes are forced to produce O₂ to use in their processes. However, by utilizing the O₂ by-product from the electrolysis process, the costs of producing O₂ can be reduced and/or eliminated.

Several C1-generating industrial processes involving partial oxidation reactions, require an O₂ input. Exemplary industrial processes include Basic Oxygen Furnace (BOF) reactions; COREX or FINEX steel making processes, Blast Furnace (BF) processes, ferroalloy production processes, titanium dioxide production processes, and gasification processes. Gasification processes include, but are not limited, to municipal solid waste gasification, biomass gasification, pet coke gasification and coal gasification. In one or more of these industrial processes, the O₂ from the carbon dioxide electrolysis process may be used to off-set or completely replace the O₂ typically supplied through air separation.

Due to the vast difference in the price of electricity in a given location, and the effect of electricity price on the efficiency of electrolysis process as a gas source for fermentation, it is largely advantageous to have a flexible approach for the utilization of electrolysis process. For example, utilizing electrolysis process as a gas source for fermentation when electricity is relatively cheap, and discontinuing use for periods of time in which prices are high. This demand-responsive utilization of electrolysis process can add tremendous value to a gas fermentation facility.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (i.e., “or”) should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (i.e., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

### EXAMPLES

The following examples further illustrate the methods and systems of the disclosure but should not be construed to limit its scope in any way.

### Example 1. Plot of the Gas Uptake Per Litre of Bioreactor Liquid Volume for the Major Gas Components Over the Course of a 25-Day Continuous C. Necator Gas Fermentation

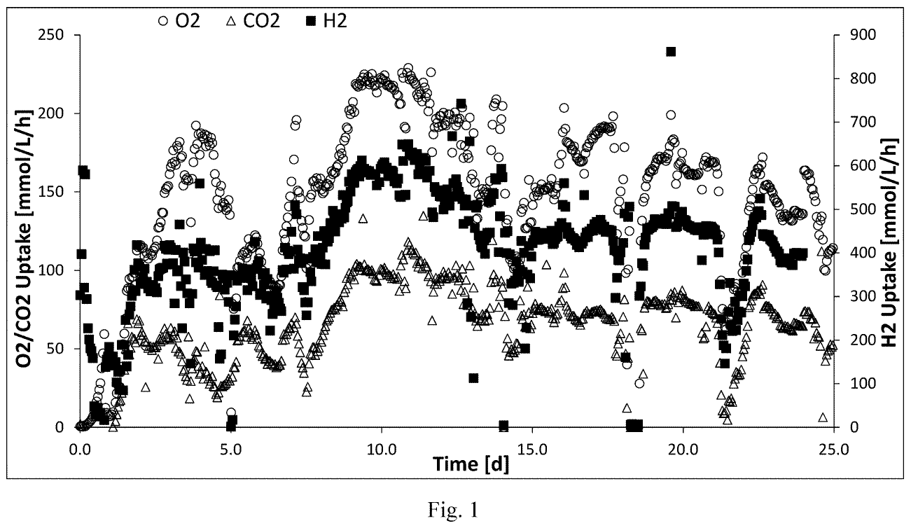

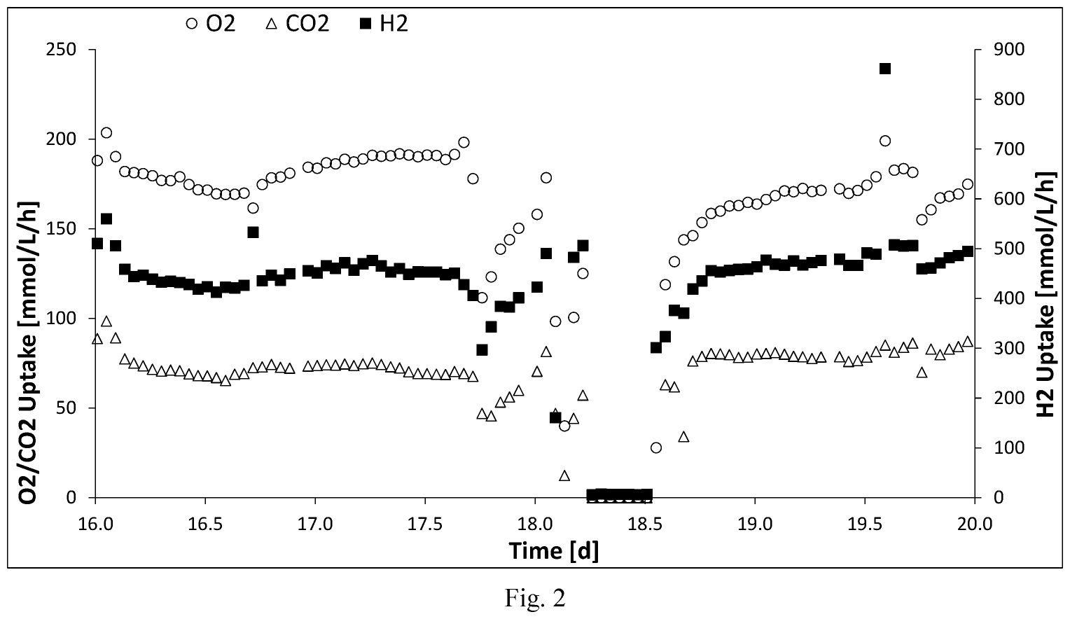

Hydrogen was the energy source and CO₂ was the carbon source. The feed gas flow was lost at day 18.21 and recovered approximately 8 hours later. There was no significant change in the long-term stability of the fermentation, any fluctuations after gas recovery were within the normal operational fluctuations for the run (FIG. 1). Hydrogen was the energy source and CO₂ was the carbon source. The gas uptake was almost immediately recovered after the resumption of the gas flow approximately 8 hours after the feed gas stopped flowing (FIG. 2).

### Example 2. Example Plot of Stable Biomass Production for a C. Necator Gas Fermentation

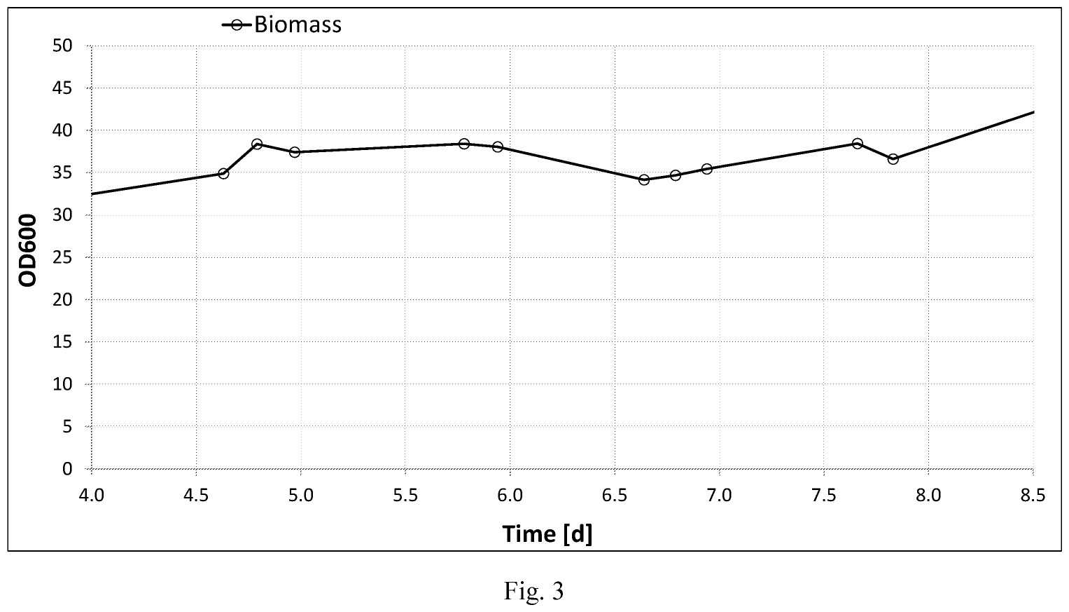

Hydrogen was shown as the energy source and CO₂ was the carbon source. The continuous stable production over a 4.5-day period with an OD600 above 30 (equivalent to ˜30 g/L DCW C. necator biomass) was seen (FIG. 3).

### Example 3. Plot of Stable Gas Uptake Per Litre of Bioreactor Liquid Volume for the Major Gas Components in a C. Necator Gas Fermentation

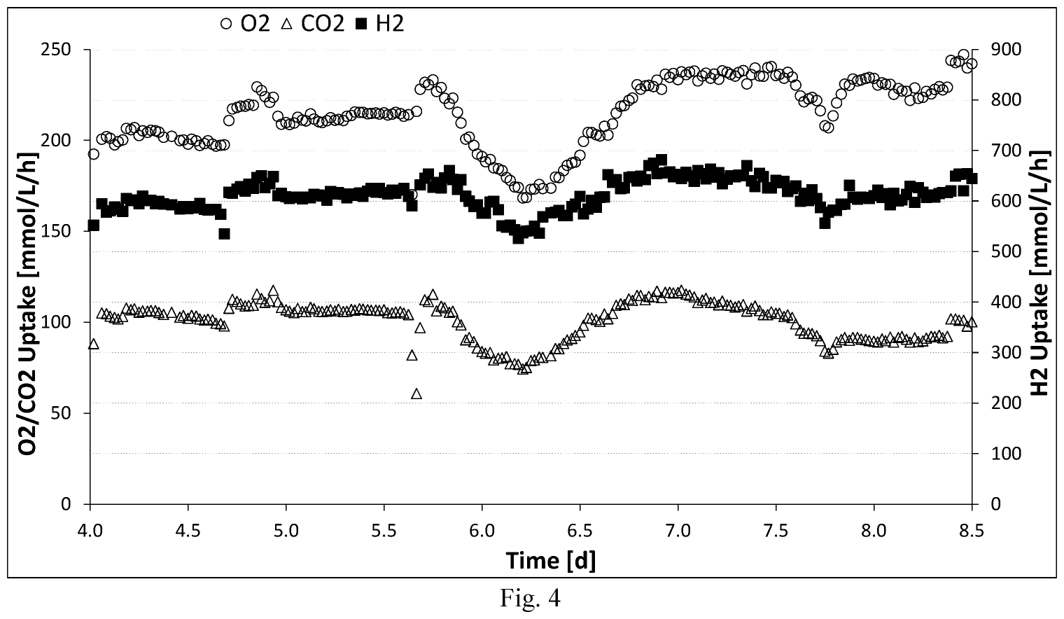

Hydrogen was the energy source and CO₂ was the carbon source. The continuous stable gas uptake over the same 4.5-day period was shown (FIG. 4).

### Example 4. A Schematic Flow Diagram Depicting the Integration of an Industrial Process and an Electrolysis Process with a Fermentation Process

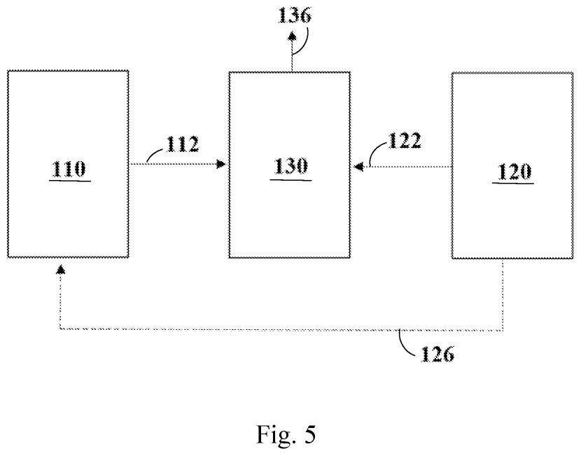

(FIG. 5) shows the integration of an industrial process 110 and an electrolysis process 120 with a fermentation process 130. The fermentation process 130 is capable of receiving C1 feedstock from the industrial process 110 and/or gases from the electrolysis process 120. The electrolysis process 120 may be fed to the fermentation process 130 intermittently. Preferably, the C1 feedstock from the industrial process 110 is fed via a conduit 112 to the fermentation process 130, and the gas from the electrolysis 120 is fed via a conduit 122 to the fermentation process 130. The fermentation process 130 utilizes the gas from the electrolysis process 110 and the C1 feedstock from the industrial process 110 to produce one or more fermentation product 136.

In certain instances, the electrolysis process comprises CO. In certain instances, the electrolysis comprises H₂. In certain instances, the gas from the electrolysis process 120 displaces at least a portion of the C1 feedstock from the industrial process 110. Preferably, the electrolysis process displaces at least a portion of the C1 feedstock as a function of the cost per unit of the C1 feedstock and the cost per unit of the electrolysis process. In various instances, the electrolysis process displaces at least a portion of the C1 feedstock when the cost per unit of electrolysis process is less than the cost per unit of C1 feedstock.

The cost per unit of electrolysis process may be less than the cost per unit of the C1 feedstock when the cost of electricity is reduced. In certain instances, the cost of electricity is reduced due to the electricity being sourced from a renewable energy source. In certain instances, the renewable energy source is selected from the group consisting of solar, hydro, wind, geothermal, biomass, nitrogen, and nuclear.

The gas from the electrolysis process 120 may supplement the C1 feedstock from the industrial process 110. Preferably, the electrolysis process supplements the C1 feedstock when the supply of the C1 feedstock is insufficient for the fermentation process. In certain instances, the electrolysis process supplements the C1 feedstock as a function of the cost per unit of the electrolysis process and the value per unit of the fermentation product 136. In certain instances, the electrolysis process supplements the C1 feedstock as a function of the cost per unit of the C1 feedstock, the cost per unit of the electrolysis process, and the value per unit of the fermentation product 136. Preferably, the gas from the electrolysis process 120 supplements the C1 feedstock when the cost per unit of the electrolysis process is less than the value per unit of the fermentation product 136. In various instances, the supplementing of the C1 feedstock comprising CO₂ with the electrolysis process comprising H₂ increases the amount of CO₂ fixed in the one or more fermentation product 136.

In one embodiment, a method for storing energy in the form of a biopolymer comprising:
a) intermittently processing at least a portion of electric energy generated from a renewable and/or non-renewable energy source in an electrolysis process to produce at least H₂, O₂ or CO;
b) intermittently passing at least one of H₂, O₂, or CO from the electrolysis process to a bioreactor containing a culture comprising a liquid nutrient medium and a microorganism capable of producing a biopolymer; and
c) fermenting the culture.

In one embodiment, wherein the electrolysis process has a cost per unit electric energy.

In one embodiment, further comprising passing a C1 feedstock comprising one or both of CO and CO₂ from an industrial or syngas process to the bioreactor, wherein the C1 feedstock has a cost per unit.

In one embodiment, wherein the biopolymer has a cost per unit.

In one embodiment, further comprising passing at least a portion of the O₂ produced in the electrolysis process to a combustion or gasification process to produce the carbon dioxide.

In one embodiment, wherein the electric energy is generated by a renewable energy source.

In one embodiment, wherein the renewable energy source comprises solar energy, wind power, wave power, tidal power, hydro power, geothermal energy, biomass and/or biofuel combustion, nuclear, or any combination thereof.

In one embodiment, wherein intermittently passing comprises any time period between continuous passing of at least one of H₂, O₂, or CO and no passing of at least one of H₂, O₂, and CO for up to about 0-2, 0-4, 0-6, 0-8, 0-10, 0-12, or 0-16 hours.

In one embodiment, wherein the electrolysis process is operated to supplement a C1 feedstock during time periods when the cost per unit electric energy is less than the cost per unit of C1 feedstock.

In one embodiment, wherein the microorganism is an autotrophic bacteria.

In one embodiment, wherein the autotrophic bacteria is Cupriavidus necator.

In one embodiment, wherein the biopolymer is a polyhydroxyalkanoate.

In one embodiment, wherein the microorganism is capable of co-producing a high nutrient protein.

In one embodiment, further comprising processing the microorganism to a generate a single cell protein (SCP) product.

In one embodiment, further comprising processing the microorganism to generate a cell-free protein synthesis platform.

In one embodiment, a system for storing energy in the form of biopolymer comprising:
a) an electrolysis process in intermittent fluid communication with a renewable and/or non-renewable energy source for producing at least one of H₂, O₂, or CO;
b) an industrial plant for producing at least C1 feedstock;
c) a bioreactor, in intermittent fluid communication with the electrolysis process and/or in continuous fluid communication with the industrial plant, comprising a reaction vessel suitable for intermittently growing, fermenting, and/or culturing and housing a microorganism capable of producing a biopolymer.

In one embodiment, further comprising at least one oxygen enriched combustion or gasification unit in fluid communication with the electrolysis process, the bioreactor, or both, the oxygen enriched combustion or gasification unit for producing carbon dioxide.

In one embodiment, further comprising at least one downstream processing system in fluid communication with the bioreactor selected from a recovery system, a purification system, an enriching system, a storage system, a recycling or further processing system for fermentation off-gas, hydrogen, water, oxygen, carbon dioxide, used medium and medium components, microorganism, or combinations thereof.

In one embodiment, further comprising a cell processing unit, in fluid communication with the bioreactor, wherein the microorganism is further processed to a single cell protein (SCP) and/or a cell-free protein synthesis platform.

In one embodiment, wherein the renewable energy source is selected from solar energy, wind power, wave power, tidal power, hydro power, geothermal energy, biomass and/or biofuel combustion, nuclear, or any combination thereof.

In one embodiment, wherein the microorganism is an autotrophic bacteria.

In one embodiment, wherein the autotrophic bacteria is Cupriavidus necator.

In one embodiment, wherein intermittent fluid communication comprises any time period between continuous passing of at least one of H₂, O₂, or CO and no passing of at least one of H₂, O₂, and CO for up to about 0-2, 0-4, 0-6, 0-8, 0-10, 0-12, or 0-16 hours.

1. A method for storing energy in the form of a biopolymer comprising:
a) intermittently processing at least a portion of electric energy generated from a renewable and/or non-renewable energy source in an electrolysis process to produce at least H₂, O₂ or CO;
b) intermittently passing at least one of H₂, O₂, or CO from the electrolysis process to a bioreactor containing a culture comprising a liquid nutrient medium and a microorganism capable of producing a biopolymer; and
c) fermenting the culture.

2. The method according to claim 1, wherein the electrolysis process has a cost per unit electric energy.

3. The method according to claim 1, further comprising passing a C1 feedstock comprising one or both of CO and CO₂ from an industrial or syngas process to the bioreactor, wherein the C1 feedstock has a cost per unit.

4. The method according to claim 1, wherein the biopolymer has a cost per unit.

5. The method according to claim 2, further comprising passing at least a portion of the O₂ produced in the electrolysis process to a combustion or gasification process to produce the carbon dioxide.

6. The method according to claim 1, wherein the electric energy is generated by a renewable energy source.

7. The method according to claim 6, wherein the renewable energy source comprises solar energy, wind power, wave power, tidal power, hydro power, geothermal energy, biomass and/or biofuel combustion, nuclear, or any combination thereof.

8. The method according to claim 1, wherein intermittently passing comprises any time period between continuous passing of at least one of H₂, O₂, or CO and no passing of at least one of H₂, O₂, and CO for up to about 0-2, 0-4, 0-6, 0-8, 0-10, 0-12, or 0-16 hours.

9. The method according claim 2, wherein the electrolysis process is operated to supplement a C1 feedstock during time periods when the cost per unit electric energy is less than the cost per unit of C1 feedstock.

10. The method according to claim 1, wherein the microorganism is an autotrophic bacteria.

11. The method according to claim 10, wherein the autotrophic bacteria is Cupriavidus necator.

12. The method according to claim 1, wherein the biopolymer is a polyhydroxyalkanoate.

13. The method according to claim 1, wherein the microorganism is capable of co-producing a high nutrient protein.

14. The method according to claim 1, further comprising processing the microorganism to a generate a single cell protein (SCP) product.

15. The method according to claim 1, further comprising processing the microorganism to generate a cell-free protein synthesis platform.

16. A system for storing energy in the form of biopolymer comprising:
a) an electrolysis process in intermittent fluid communication with a renewable and/or non-renewable energy source for producing at least one of H₂, O₂, or CO;
b) an industrial plant for producing at least C1 feedstock;
c) a bioreactor, in intermittent fluid communication with the electrolysis process and/or in continuous fluid communication with the industrial plant, comprising a reaction vessel suitable for intermittently growing, fermenting, and/or culturing and housing a microorganism capable of producing a biopolymer.

17. The system according to claim 16, further comprising at least one oxygen enriched combustion or gasification unit in fluid communication with the electrolysis process, the bioreactor, or both, the oxygen enriched combustion or gasification unit for producing carbon dioxide.

18. The system according to claim 16, further comprising at least one downstream processing system in fluid communication with the bioreactor selected from a recovery system, a purification system, an enriching system, a storage system, a recycling or further processing system for fermentation off-gas, hydrogen, water, oxygen, carbon dioxide, used medium and medium components, microorganism, or combinations thereof.

19. The system according to claim 16, further comprising a cell processing unit, in fluid communication with the bioreactor, wherein the microorganism is further processed to a single cell protein (SCP) and/or a cell-free protein synthesis platform.

20. The system according to claim 16, wherein the renewable energy source is selected from solar energy, wind power, wave power, tidal power, hydro power, geothermal energy, biomass and/or biofuel combustion, nuclear, or any combination thereof.

21. The system according to claim 16, wherein the microorganism is an autotrophic bacteria.

22. The system according to claim 21, wherein the autotrophic bacteria is Cupriavidus necator.

23. The system according to claim 16, wherein intermittent fluid communication comprises any time period between continuous passing of at least one of H₂, O₂, or CO and no passing of at least one of H₂, O₂, and CO for up to about 0-2, 0-4, 0-6, 0-8, 0-10, 0-12, or 0-16 hours.
